(19) United States
(12) Patent Application Publication
(10) Pub. No.: US 2025/0241841 A1
(43) Pub. Date: Jul. 31, 2025

(54) POLYVINYL ALCOHOL FILM-FORMING POLYMERS FOR ALCOHOL-BASED HAIR FIXING FORMULATIONS AND METHODS OF USING SAME

(71) Applicant: NOURYON CHEMICALS INTERNATIONAL B.V., Amsterdam (NL)

(72) Inventors: Hanamanthsa Shankarsa BEVINAKATTI, Somerset, NJ (US); John Socrates THOMAIDES, Berkeley Heights, NJ (US); Qiwei HE, Belle Mead, NJ (US)

(73) Assignee: NOURYON CHEMICALS INTERNATIONAL B.V., Amsterdam (NL)

(21) Appl. No.: 18/878,360

(22) PCT Filed: Jun. 26, 2023
(86) PCT No.: PCT/EP2023/067252

§ 371 Date: Dec. 23, 2024

Related U.S. Application Data
(60) Provisional application No. 63/355,177, filed on Jun. 24, 2022.

(51) Int. Cl. A61K 8/81; A61K 8/04; A61K 8/33; A61Q 5/06

(52) U.S. Cl. CPC A61K 8/8129; A61K 8/046; A61K 8/33; A61K 8/8164; A61Q 5/06; A61K 2800/31; A61K 2800/95

(57) ABSTRACT

The present disclosure relates to a hair fixing formulation comprising: (a) a volatile solvent; and (b) at least one film-forming polymer comprising at least one of: (i) polyvinyl alcohol or (ii) polyvinyl alcohol ester said polyvinyl alcohol ester having ester functionality other than solely acetate, and (i) and (ii) being soluble in the volatile solvent, provided that if the at least one film-forming polymer comprises polyvinyl alcohol, then the polyvinyl alcohol has a degree of hydrolysis less than about 60%. Methods of using the hair fixing formulations to fix hair are also disclosed.

### CROSS-REFERENCE TO RELATED APPLICATIONS

This application is a U.S. National-Stage entry under 35 U.S.C. § 371 based on International Application No. PCT/EP2023/067252, filed Jun. 26, 2023, which was published under PCT Article 21(2) and which claims priority of U.S. Provisional Patent Application No. 63/355,177, filed Jun. 24, 2022, the entire contents of each patent application are hereby incorporated herein by reference.

### FIELD OF THE DISCLOSURE

The present disclosure relates to film-forming polymers for hair fixing formulations and to methods of using the hair fixing formulations.

### BACKGROUND

Polymers used in hair fixative formulations have conventionally been made using synthetic materials. In order for the polymers to be suitable in such hair fixative applications, they should be soluble in alcohol-based systems and/or systems containing low amounts of volatile organic compounds (VOC), and in the case of aerosol-based hair care sprays, they should also be compatible with the propellant. Conventional synthetic polymers are generally inexpensive and provide acceptable performance. However, because they are not biodegradable, conventional synthetic polymers are not sustainable. In addition, replicating the cost and performance of synthetic polymers is not easy.

Accordingly, there is a need for biodegradable film-forming polymers that provide equal to or better performance at comparable costs than the non-biodegradable alternatives and that are soluble in alcohol-based systems, such as ethanol-based systems and optionally, that are compatible with propellants, such as hydrocarbons or dimethyl ether.

Our U.S. Pat. No. 11,135,148 describes hair fixatives made from renewable sources including starch ester-based polyglucose polymers.

Suncare formulations based on biodegradable polyesters are the subject of our recent publications: US 2021/0259930; US 2021/0259945; and US 2021/0259946. Do we need this sentence here? It refers to suncare formulations,

U.S. Pat. No. 6,602,489 and WO 2006/018328 describe compact hair spray products comprising a film-forming polymer, which could be a polyvinyl alcohol sold by Kuraray under the tradename Poval™. The available Poval™ products have DH>99%. Kuraray usually provides these materials at certain viscosities without any indication of molecular weight. For example, Poval™ LM-10 HD is supplied at a viscosity of 4.5-5.7 MPa·s. Poval™ LM-20 is supplied at a viscosity of 3.0-4.0 MPa·s. Interestingly, however, Example 1 of U.S. Pat. No. 7,939,582 provides the number average molecular weight of Poval™ LM-20 as about 20,000, but it is not known if this is accurate. The polyvinyl alcohol could also be those available from Sekisui, for example, those products available under the tradenames Selvol™ PVOH 523/E 523 and Selvol™ PVOH 540, both of which have a DH of 88%.

US 2015/0328130 describes a sunscreen composition comprising 0.1 to 10% of a non-linear polymer selected from a group that includes polyvinyl alcohol; and 40 to 80% water. The exemplified compositions include various polyvinyl alcohols having degrees of hydrolysis ranging from 86-99%; and at least 70% water; but do not comprise ethanol.

AU 2017/204073 describes cosmetic compositions, in particular, sunscreens, spiked with polyvinyl alcohol to facilitate washing UV filters out of textiles contaminated with these compositions. According to the teachings, the addition of polyvinyl alcohol to the compositions has an advantageous effect on washability, reducing textile staining caused by the compositions. The effect is demonstrated with polyvinyl alcohol having a degree of hydrolysis of 86 to 89 mol %.

U.S. Pat. No. 3,417,180 describes hair spray formulations comprising polyvinyl acetates partially hydrolyzed to the extent of about 15 to 60% of their acetate groups. Solubility up to 50% is demonstrated in anhydrous ethanol and up to 48% in halocarbon propellant, particularly trichlorofluoromethane and dichlorodifluoromethane. The use of hydrocarbon propellants and ethers is neither taught nor suggested, nor is the solubility of the partially hydrolyzed polyvinyl acetates in such propellants demonstrated. Copolymers of partially hydrolyzed polyvinyl acetate and other monomers, including (meth)acrylic acid and crotonic acid are theorized but not exemplified.

U.S. Pat. No. 3,005,809 describes sheets or films of vinyl alcohol-crotonic acid copolymers.

The compatibility of film-forming polymers with alcohol-based, especially ethanol-based hair fixing formulations, remains a significant problem for the formulator and there is a significant need for film-forming polymers that are biodegradable yet at the same time are soluble in and compatible with alcohol-based systems.

Accordingly, it is an object of the present disclosure to provide film-forming polymers that are biodegradable, exhibit excellent compatibility with alcohol-based formulations, especially ethanol-based formulations, and, therefore, can be formulated into stable hair fixing formulations that provide biodegradability. In addition, other objects, desirable features and characteristics will become apparent from the subsequent summary and detailed description, and the appended claims, taken in conjunction with the accompanying drawings and this background.

### BRIEF SUMMARY

The present disclosure relates in one embodiment to a hair fixing formulation comprising:
(a) a volatile solvent; and
(b) at least one film-forming polymer comprising at least one of: (i) polyvinyl alcohol or (ii) polyvinyl alcohol ester, said polyvinyl alcohol ester having ester functionality other than solely acetate, and (i) and (ii) being soluble in the volatile solvent, provided that if the at least one film-forming polymer comprises polyvinyl alcohol, then the polyvinyl alcohol has a degree of hydrolysis less than about 60%.

The present disclosure relates in a second embodiment to a method of fixing hair comprising spraying hair with the hair fixing formulation described herein.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description. It is to be appreciated that all numerical values as provided herein, save for the actual examples, are approximate values with endpoints or particular values intended to be read as “about” or “approximately” the value as recited.

Polyvinyl alcohol (“PVOH”) has the generalized structure:

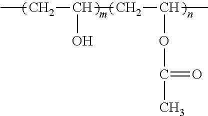

and is exemplified by pendent OH and acetate (CH₃—CO—) groups/functionality.

There are several methods known for the manufacturing of PVOH. However, from an industrial point of view, the current mainstream method of production is as follows: Polyvinyl acetate is manufactured by radical polymerization using vinyl acetate as a raw material and then the polyvinyl acetate is hydrolyzed/transesterified. Methanol or ethanol are mainly used as a solvent in the polymerization process/hydrolysis process.

The basic properties of PVOH are typically governed by the degree of polymerization and the degree of hydrolysis.

The degree of polymerization of PVOH is generally indicated by the viscosity-average degree of polymerization derived from the viscosity in water. Additionally, when expressing the degree of hydrolysis using the repeating units m and n, the degree of hydrolysis can be expressed as follows: Degree of hydrolysis (mol %)=m/(n+m)×100. This is the value with which the proportion of the vinyl alcohol unit in all the repeating units is indicated in mol %.

We have discovered that the degree of hydrolysis is an important parameter with respect to the solubility of the base polyvinyl alcohol in the volatile solvent. On the other hand, when the base polyvinyl alcohol is derivatized with hydrophobic functional groups, the degree of hydrolysis is not as important.

In one embodiment, the film-forming polymer comprises underivatized polyvinyl alcohol (referred to herein alternatively as “base polyvinyl alcohol”).

In a preferred embodiment, the base polyvinyl alcohol has a degree of hydrolysis of 60% or less, especially 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and/or 60%; or a continuous sequence of percentages within this range, for example, 1% to 60%, 1% to 59%, 2% to 59%, 2% to 58%, 3% to 58%, etc.

In another preferred embodiment, the base polyvinyl alcohol has a degree of hydrolysis of 20% to 58%, especially 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, and/or 58%; or a continuous sequence of percentages within this range, for example, 20% to 58%, 21% to 58%, 21% to 57%, 22% to 57%, 22% to 56%, etc.

In another preferred embodiment, the base polyvinyl alcohol has a degree of hydrolysis of 35% to 58%, especially 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, and/or 58%; or a continuous sequence of percentages within this range, for example, 35% to 58%, 36% to 58%, 36% to 57%, 37% to 57%, 37% to 56%, etc.

In the case of a range qualified with the term “about,” for example, “less than about 60%” or “about 35% to about 58%,” the range is to be expanded a maximum of 1%, preferably at most 0.5%.

Once the base PVOH has been prepared, the base PVOH can be esterified by further reaction with a suitable functionalizing agent, for example, an acid, an anhydride, or a lactone, according to methods known in the art. It is also possible to prepare the ester starting with vinyl acetate and other vinyl monomers at specified ratios to form an intermediate copolymer having pendent acetate functionality and then hydrolyzing the acetate group selectively to give desired PVOH portion.

Thus, in another embodiment, the at least one film-forming polymer comprises polyvinyl alcohol derivatized with additional ester functionality different from the pendent OH and acetate groups that exemplify the base polyvinyl alcohol.

As used herein, simply “polyvinyl alcohol” or “PVOH” without more, or “base polyvinyl alcohol” or “base PVOH” means polyvinyl alcohol including only pendent OH and acetate groups.

As used herein, “polyvinyl alcohol esters” or “PVOH esters” or the like means polyvinyl alcohol derivatized with additional ester functionality different from the pendent OH and acetate groups that exemplify the base polyvinyl alcohol.

As noted above, one way PVOH esters can be formed is by reacting the base PVOH with a suitable acid or a suitable acid derivative, such as an anhydride or lactone.

Suitable acids include linear, branched or cyclic, saturated or unsaturated, nonaromatic or aromatic monocarboxylic acids or polycarboxylic acids.

Exemplary acids useful for this purpose include, without being limiting, propionic acid, butyric acid, valeric acid, isovaleric acid, caproic acid, heptanoic acid, octanoic acid, nonanoic acid, decanoic acid, dodecanoic acid, octadecanoic acid, pivalic acid, 2-ethylhexanoic acid, isononanoic acid, neononanoic acid, neodecanoic acid, Versatic Acid 9 (highly branched nonanoic acid available from Hexion), Versatic Acid 10 (highly branched decanoic acid available from Hexion), palmitic acid, benzoic acid, anthranilic acid, salicylic acid, phenyl acetic acid, cinnamic acid, 4-chlorobenzoic acid, 1,4-dichlorobenzoic acid, 4-nitrobenzoic acid, 2,4-dinitrobenzoic acid, succinic acid, glutaric acid, adipic acid, octanedioic acid (suberic acid), malonic acid, malic acid, octenyl succinic acid, dodecenyl succinic acid, azelaic acid, sebacic acid, pimelic acid, 2,2-dimethylsuccinic acid, 3,3-dimethylglutaric acid, 2,2-dimethylglutaric acid, maleic acid, fumaric acid, itaconic acid, n-enanthic acid, n-caprylic acid, n-pelargonic acid, n-capric acid, n-lauric acid, n-myristic acid, n-stearic acid, oleic acid, oxalic acid, malonic acid, pimelic acid, azelaic acid, sebacic acid, benzoic acid, phthalic acid, citric acid, tartaric acid, lactic acid, malic acid, toluic acid, isophthalic acid, phthalic acid, terephthalic acid, trimellitic acid, 1,2-cyclohexane dicarboxylic acid, 1,3-cyclohexane dicarboxylic acid, tetrahydrophthalic acid, endomethylene tetrahydrophthalic acid, hexahydrophthalic acid, tetrachlorophthalic acid, methyl hexahydrophthalic acid, substituted derivatives thereof, as well as mixtures of one or more of the foregoing.

Anhydrides of the above acids, where they exist, can also be employed, such as succinic anhydride, dimethyl ester and diethyl ester of malonic acid, octenyl succinic anhydride, dodecenyl succinic anhydride, phthalic anhydride, trimellitic anhydride, tetrahydrophthalic anhydride, endomethylene tetrahydrophthalic anhydride, hexahydrophthalic anhydride, methyl hexahydrophthalic anhydride.

In addition, certain materials that react in a manner similar to acids to form esters are also useful. Such materials include lactones, for example, caprolactone, propylolactone, methyl caprolactone, and butyrolactone.

As also noted above, PVOH esters can be made by free-radical polymerization of vinyl acetate and other vinyl monomers at specified ratios to form an intermediate copolymer having pendent acetate functionality and then hydrolyzing the acetate group selectively to give desired PVOH portion. Polymerization methods known in the art such as but not limited to solution polymerization, bulk polymerization, precipitation polymerization, emulsion polymerization, and suspension polymerization can be used to polymerize vinyl acetate with other vinyl monomers. In one preferred embodiment, the polymerization is a solution polymerization. In another preferred embodiment, the polymerization is a suspension polymerization.

The disclosed hair fixing formulations additionally comprise a volatile solvent. Examples of volatile solvents include one or more of alcohols, such as methanol, ethanol and isopropanol; volatile hydrocarbons, such as isooctane, isododecane, and isohexadecane; volatile aldehydes; volatile silicones; and volatile ketones, such as acetone and methyl ethyl ketone. Hydrofluoro-olefins may also be used as a carrier solvent in the formulations.

In a preferred embodiment, the volatile solvent is an alcohol-based solvent system, wherein the alcohol-based solvent system comprises at least one C₁₋₆ straight or branched chain alcohol.

In another preferred embodiment, the volatile solvent is a ketone.

In an especially preferred embodiment, the volatile solvent is chosen from the group consisting of ethanol, methanol, isopropanol, acetone, and mixtures thereof.

In another especially preferred embodiment, the hair fixing formulations comprise ethanol.

In a more preferred embodiment, the hair fixing formulations comprising ethanol are anhydrous. The terms “non-aqueous” and “anhydrous” are used interchangeably herein and refer to compositions containing less than about 10% by weight water, especially less than about 5% by weight water, or less than 1% by weight water, or even 0% water.

In a most preferred embodiment, the hair fixing formulation comprises ethanol but not any other alcohol or water.

In an alternative embodiment, where water is present, this small amount of water may be desirable, for example as a processing aid or co-solvent. In certain example embodiments, the water contents of the compositions will be no greater than about 9% water so as to prevent the active to phase-separate or precipitate out of solution. Those of ordinary skill in the art will recognize that different actives have different tolerances for water in solution and will adjust water content accordingly.

In an embodiment, the polymer is fully soluble in an ethanol or a predominately ethanol mixture with 0-20 wt %, preferably 5-10% water.

In another embodiment, the polymer is fully soluble in ethanol.

In another embodiment, the polymer is fully soluble in anhydrous ethanol.

In another embodiment, the polymer is fully soluble in anhydrous ethanol immediately after the polymer is incorporated into the solvent. The phrase “immediately after” as used throughout this disclosure means an hour or less, preferably a half-hour or less, most preferably 15 minutes or less.

In another embodiment, the polymer remains fully soluble in anhydrous ethanol after 24 hours after the polymer is incorporated into the solvent.

In an especially preferred embodiment, the polymer is:
(a) fully soluble in anhydrous ethanol immediately after the polymer is incorporated into the solvent; and
(b) fully soluble in anhydrous ethanol after 24 hours after the polymer is incorporated into the solvent.

In an even more preferred embodiment, the polymer comprises polyvinyl alcohol or an ester thereof, wherein if the polymer comprises underivatized polyvinyl alcohol, the polyvinyl alcohol has a degree of hydrolysis of 35-58% percent and is:
(a) fully soluble in anhydrous ethanol immediately after the polymer is incorporated into the solvent; and
(b) fully soluble in anhydrous ethanol after 24 hours after the polymer is incorporated into the solvent.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-45% and is:
(a) fully soluble in anhydrous ethanol immediately after the polymer is incorporated into the solvent; and
(b) fully soluble in anhydrous ethanol after 24 hours after the polymer is incorporated into the solvent.

In an even more preferred embodiment, the polymer comprises polyvinyl alcohol or an ester thereof, wherein if the polymer comprises underivatized polyvinyl alcohol, the polyvinyl alcohol has a degree of hydrolysis of 38-42% percent and is:
(a) fully soluble in anhydrous ethanol immediately after the polymer is incorporated into the solvent; and
(b) fully soluble in anhydrous ethanol after 24 hours after the polymer is incorporated into the solvent.

In one embodiment, the hair fixing formulations further comprise a propellant.

In certain embodiments of the subject disclosure, the disclosed formulations can be stored in containers under pressure by combination with the propellant and the disclosure extends to such containers under pressure containing the disclosed formulations. The formulations thus stored can be applied by opening a valve in the container releasing the propellant and the composition, typically in a spray or mist. The propellant used in the composition may be any suitable gas, or combination of gasses, that can be compressed or liquefied within a dispensing spray canister, which expand or volatilize to vapor or gas form upon exposure to ambient temperature and pressure conditions to deliver the composition in an aerosol form. Suitable propellants include hydrocarbons having 1 to 5 carbon atoms, including but not limited to methane, ethane, propane, isopropane, butane, isobutane, butene, pentane, isopentane, neopentane, and pentene, hydrofluorocarbons (HFCs), including but not limited to 1,1-difluoroethane (HP 152a), chlorofluorocarbons (CFCs), hydrofluoro-olefins (HFOs), nitrogen, ethers including dimethyl ether, and any mixtures thereof. Those of ordinary skill in the art recognize that in a closed container such as an aluminum can or glass bottle, propellants such as dimethyl ether condense to the liquid state at ambient temperature. Thus, the composition in the aerosol container is a liquid formulation that can contain dissolved propellant, undissolved liquid propellant and gaseous propellant. All of this is under pressure due to the vapor pressure of the propellant. In the practice of this aspect of the subject disclosure, the propellant can be present in an amount up to about 90 weight percent, preferably from about 2 weight percent to about 50 weight percent, and more preferably about 5 weight percent to about 40 weight percent, more preferably at about 30 weight percent, based on the total weight of the aerosol composition.

In another embodiment, the propellant is a hydrocarbon-derived ether.

In another embodiment, the propellant is dimethyl ether (DME).

In a preferred embodiment, the polymer is fully compatible with DME.

In an especially preferred embodiment, the polymer is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In an even more preferred embodiment, the polymer comprises polyvinyl alcohol or an ester thereof, wherein if the polymer comprises underivatized polyvinyl alcohol, the polyvinyl alcohol has a degree of hydrolysis of 35-58% percent and is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-45% and is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In an even more preferred embodiment, the polymer comprises polyvinyl alcohol or an ester thereof, wherein if the polymer comprises underivatized polyvinyl alcohol, the polyvinyl alcohol has a degree of hydrolysis of 38-42% percent and is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with DME.

In another embodiment, the propellant is a hydrofluorocarbon.

In another embodiment, the propellant is 1,1-difluoroethane.

In a preferred embodiment, the polymer is fully compatible with 1,1-difluoroethane.

In an especially preferred embodiment, the polymer is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-58% and is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In a more preferred embodiment, the polymer comprises polyvinyl alcohol or an ester thereof, wherein if the polymer comprises underivatized polyvinyl alcohol, the polyvinyl alcohol has a degree of hydrolysis of 35-45% and is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In an even more preferred embodiment, the polymer comprises polyvinyl alcohol or an ester thereof, wherein if the polymer comprises underivatized polyvinyl alcohol, the polyvinyl alcohol has a degree of hydrolysis of 38-42% percent and is:
(a) fully soluble in anhydrous ethanol immediately after the polymer is incorporated into the solvent;
(b) fully soluble in anhydrous ethanol after 24 hours after the polymer is incorporated into the solvent; and
(c) fully compatible with 1,1-difluoroethane.

In one embodiment, the hair fixing formulation disclosed here is dispensed from a bag-on-valve device. Generally, bag-on-valve devices comprise a spray can fitted with an aerosol valve and comprising a welded bag. The product is placed inside the bag while the propellant is filled in the space between the bag and the can. The product is dispensed by the propellant simply squeezing the bag when the spray button is pressed.

Generally, the compositions according to the disclosure are prepared as non-aqueous, volatile solvent-based compositions. However, in some embodiments, the compositions comprise a single liquid phase that may further comprise dispersed particulates, for example, UV active agents in particulate form.

We have found that in certain instances the base polyvinyl alcohol polymer as described herein above, which contains predominantly only pendent alcohol and acetate groups, can be derivatized without loss of solubility in the volatile solvent.

The solubility of the derivatized polyvinyl alcohol in such volatile solvent can be assessed by using the testing protocols set forth in the examples hereinbelow.

As noted above, the main production method for producing polyvinyl alcohol involves manufacturing polyvinyl acetate by radical polymerization using vinyl acetate as a raw material and then hydrolyzing the polyvinyl acetate. Derivatives can then be made by, for example, (a) reacting polyvinyl alcohol with an acid, an anhydride, or a lactone; or (b) directly by (i) polymerizing a polymerizable monomer precursor of the derivatizing functional group, for example, a vinyl alkanoate, along with the vinyl acetate followed by (ii) hydrolysis to create alcohol functionality in any desired degree of hydrolysis.

In one embodiment, the polyvinyl alcohol is derivatized with crotonic acid, for example, by copolymerizing crotonic acid with vinyl acetate, the product of which is then at least partially hydrolyzed/transesterified to yield the desired polyvinyl alcohol derivative. In a preferred embodiment, the product is a partially hydrolyzed vinyl acetate-crotonic acid copolymer comprising pendant carboxylic acid groups and pendant hydroxy groups condensed together to form an internal gamma-lactone functionality.

In one embodiment, the polyvinyl alcohol polymer is derivatized with succinic acid, for example, by reacting a polyvinyl alcohol soluble in the volatile solvent with succinic anhydride.

In another embodiment, the polyvinyl alcohol polymer is derivatized with caprolactone, for example, by reacting a polyvinyl alcohol soluble in the volatile solvent with caprolactone.

In another embodiment, the polyvinyl alcohol polymer is derivatized with pivalic acid, for example, by copolymerizing vinyl pivalate with vinyl acetate, the product of which is then hydrolyzed/transesterified to yield the desired polyvinyl alcohol derivative.

In another embodiment, the polyvinyl alcohol polymer is derivatized with neononanoic acid, for example, by copolymerizing vinyl neononanoate with vinyl acetate, the product of which is then hydrolyzed/transesterified to yield the desired polyvinyl alcohol derivative.

In another embodiment, the polyvinyl alcohol polymer is derivatized with Versatic acid 9, for example, by copolymerizing the vinyl ester of Versatic acid 9 (e.g., VeoVa 9 from Hexion) with vinyl acetate, the product of which is then hydrolyzed/transesterified to yield the desired polyvinyl alcohol derivative.

In another embodiment, the polyvinyl alcohol polymer is derivatized with neodecanoic acid, for example, by copolymerizing vinyl neodecanoate with vinyl acetate, the product of which is then hydrolyzed/transesterified to yield the desired polyvinyl alcohol derivative.

In another embodiment, the polyvinyl alcohol polymer is derivatized with Versatic Acid 10, for example, by copolymerizing vinyl neodecanoate (e.g., VeoVa 10 from Hexion) with vinyl acetate, the product of which is then hydrolyzed/transesterified to yield the desired polyvinyl alcohol derivative.

In another embodiment, the polyvinyl alcohol polymer is derivatized with 2-ethylhexanoic acid, for example, by polymerizing vinyl 2-ethylhexanoate with vinyl acetate, the product of which is then hydrolyzed/transesterified to yield the desired polyvinyl alcohol derivative.

In another embodiment, the polyvinyl alcohol polymer is derivatized with lauric acid, for example, by polymerizing vinyl laurate with vinyl acetate, the product of which is then hydrolyzed/transesterified to yield the desired polyvinyl alcohol derivative.

In another embodiment, the poly(vinyl alcohol) polymer is derivatized with a carboxylic acid pendent from the polymer backbone yielding an internal lactone (cyclic ester) moiety.

In a preferred embodiment, the carboxylic acid pendent from the polymer backbone reacts with an alcohol pendent from the gamma-carbon relative to the carbon of the pendent carboxylic acid to form an internal gamma-lactone (five-membered ring).

In a preferred embodiment, the carboxylic acid pendent from the polymer backbone is the residue of acrylic acid or crotonic acid obtained after copolymerization of acrylic acid or crotonic acid, respectively, with vinyl acetate and, optionally, one or more vinyl alkanoates followed by hydrolysis to create alcohol functionality in any desired degree of hydrolysis.

In a more preferred embodiment, the carboxylic acid pendent from the polymer backbone is the residue of crotonic acid obtained after copolymerization of crotonic acid with vinyl acetate and, optionally, one or more vinyl alkanoates followed by hydrolysis to create alcohol functionality in any desired degree of hydrolysis.

In a most preferred embodiment, the carboxylic acid pendent from the polymer backbone is the residue of crotonic acid obtained after copolymerization of crotonic acid with vinyl acetate and, optionally, one or more vinyl alkanoates followed by hydrolysis to create alcohol functionality in any desired degree of hydrolysis and the internal lactone formed is a gamma-lactone.

In an especially preferred embodiment, the polyvinyl alcohol is derivatized with 1-15 wt % crotonic acid gamma-lactone based on 100 wt % of the polyvinyl alcohol polymer.

In a more preferred embodiment, the polyvinyl alcohol is derivatized with 2-13 wt % crotonic acid gamma-lactone based on 100 wt % of the polyvinyl alcohol polymer.

In an even more preferred embodiment, the polyvinyl alcohol is derivatized with 6-12 wt % crotonic acid gamma-lactone based on 100 wt % of the polyvinyl alcohol polymer.

In a most preferred embodiment, the polyvinyl alcohol is derivatized with 6-12 wt % crotonic acid gamma-lactone based on 100 wt % of the polyvinyl alcohol polymer, which has a degree of hydrolysis of 35-50%.

It should be noted that the carboxylic acid pendent from the polymer that is the residue of acrylic acid or crotonic acid obtained after copolymerization of acrylic acid or crotonic acid, respectively, with vinyl acetate and, optionally, one or more vinyl alkanoates followed by hydrolysis to create alcohol functionality in any desired degree of hydrolysis is known to form a lactone with the alcohol functionality pendent from the polyvinyl alcohol polymer. For this reason, steps are sometimes taken to inhibit the formation of the lactone. However, we have discovered that surprisingly and unexpectedly the lactone form is beneficial particularly in the context of providing high humidity curl retention (HHCR).

The hair fixing formulation may additionally comprise other film-forming polymers in addition to the polyvinyl alcohol described herein, especially, but not only where the intended use is in hair care. Such other film-forming polymers can be selected from, merely for example, starch ester-based polyglucose polymers, such as are described in U.S. Pat. No. 11,135,148, the entire contents of which are hereby incorporated by reference; polyesters, such as are described in US 2021/0259930, US 2021/0259945, and US 2021/0259946, the entire contents of which are hereby incorporated by reference; and N-alkyl (meth)acrylamide copolymers, such as are described in US 20180098930, the entire contents of which are hereby incorporated by reference. Additional examples of suitable other film-forming polymers include film-forming polymer comprising at least 5% by weight, based on a total weight of the film-forming polymer, of an acid-containing monomer. In a preferred embodiment, the film-forming polymer comprises at least 5% by weight of a carboxylic acid-containing monomer. Non-limiting examples of these monomers are acrylic acid, crotonic acid, methacrylic acid, maleic acid, itaconic acid, and combinations and mixtures thereof. Additional film-forming polymers, either synthetic or natural can be used with the acid-containing polymers described above. Non-limiting examples of these additional film forming polymers are: from Nouryon, AMPHOMER® and AMPHOMER® LV-71 polymers (octylacrylamide/acrylates/butylaminoethyl methacrylate com polymer), AMPHOMER® HC® polymer (acrylates/octylacrylamide copolymer), BALANCE® 0/55 and BALANCE CR® polymers (acrylates copolymer), BALANCE® 47 polymer (octylacrylamide/butylaminoethyl methacrylate copolymer), RESYN® 28-2930 polymer (VA/crotonates/vinyl neodecanoate copolymer), RESYN® 28-1310 polymer (VA/Crotonates copolymer), FLEXAN® polymers (sodium polystyrene sulfonate), DynamX polymer (polyurethane-14 (and) AMP-Acrylates copolymer), RESYN XP® polymer (acrylates/octylacrylamide copolymer), STRUCTURE 2001 (acrylates/steareth-20 itaconate copolymer) and STRUCTURE® 3001 (acrylates/ceteth-20 itaconate copolymer); from ISP, OMNIREZ-2000® (PVM/MA half ethyl ester copolymer), GANEX P-904® (butylated PVP), GANEX V-216® (PVP/hexadecene copolymer) GANEX® V-220 (PVP/eicosene copolymer), GANEX® WP-660 (tricontanyl PVP), GANTREZ® A425 (butyl ester of PVM/MA copolymer), GANTREZ® AN-119 PVM/MA copolymer, GANTREZ ES 225® (ethyl ester of PVM/MA copolymer), GANTREZ ES425 (butyl ester of PVM/MA copolymer), GAFFIX VC-713® (vinyl caprolactam/PVP/dimethylaminoethyl methacrylate copolymer), GAFQUAT755® (polyquaternium-11), GAFQUAT HS-100® (poly-quaternium-28), AQUAFLEX XL-306 (Polyimide-1), AQUAFLEX SF-40® (PVP/Vinylcaprolactam/DMAPA Acrylates Copolymer), AQUAFLEX FX-64® (Isobutylene/Ethylmaleimide/Hydroxyethylmaleimide Copolymer), ALLIANZ LT-120® (Acrylates/C1-2 Succinates/Hydroxy acrylates Copolymer), STYLEZE CC-10® (PVP/DMAPA Acrylates Copolymer), STYLEZE 2000® (VP/Acrylates/Lauryl Methacrylate Copolymer), STYLEZE W-20® (Poly quaternium-55), Copolymer Series (PVP/Dimethylamino ethylmethacrylate Copolymer), ADVANTAGE S® and ADVANTAGE LCA® (VinylcaprolactamNP/Dimethylaminoethyl Methacrylate Copolymer), ADVANTAGE PLUS (VA/Butyl Maleate/Isobornyl Acrylate Copolymer), Antaron ECo (Ethylcellulose); from BASF, ULTRAHOLD STRONG (acrylic acid/ethyl acrylate/t-butyl acrylamide), LUVIMER 100P® (t-butyl acrylate/ethyl acrylate/methacrylic acid), LUVIMER 36D (ethyl acrylate/t-butyl acrylate/methacrylic acid), LUVIQUAT HM-552® (polyquaternium-16), LUVIQUAT HOLD® (polyquaternium-16), LUVISKOL K30 (PVP) LUVISKOL K90® (PVP), LUVISKOL VA 64® (PVP/VA copolymer), LUVISKOL VA73W® (PVPNA copolymer), LUVISKOL VA®, LUVISET PUR® (Polyurethane-1), LUVISET® Clear (VP/MethacrylamideNinyl Imidazole Copolymer), LUVIFLEX SOFT® (Acrylates Copolymer), ULTRA HOLD 8® (Acrylates/Acrylamide Copolymer), LUVISKOL® Plus (Polyvinylcaprolactam), LUVIFLEX® Silk (PEG/PPG-25/25 Dimethicone/Acrylates Copolymer); from Amerchol, AMERHOLD® DR-25 (acrylic acid/meth acrylic acid/acrylates/methacrylates); from Rohm & Haas, ACUDYNE 258® (acrylic acid/methacrylic acid/acrylates/methacrylates/hydroxy ester acrylates; from Mitsubishi and distributed by Clariant, DIAFORMER Z-301®, DIA FORMER Z-SM®, and DIAFORMER Z-400® (methacryloyl ethyl betaine/acrylates copolymer), ACUDYNE 180® (Acrylates/Hydroxyesters Acrylates Copolymer), ACU DYNE SCP (Ethylenecarboxyamide/AMPSA/Methacrylates Copolymer), and the ACULYN® rheological modifiers; from ONDEO Nalco, FIXOMER A-30® and FIXOMER N-28 ® (INCI names: methacrylic acid/sodium acrylamidomethyl propane sulfonate copolymer); from Noveon, FIXATE G-100® (AMP-Acrylates/Allyl Meth acrylate Copolymer), FIXATE PLUS® (Polyacrylates-X), CARBOPOL® Ultrez 10 (Carbomer), CARBOPOL Ultrez 20® (Acrylates/C10-30 Alkyl Acrylates Copolymer), AVALLTRE AC® series (Acrylates Copolymer), AVALURE UR® series (Polyurethane-2, Polyurethane-4, PPG-17/IPDI/DMPA Copolymer); polyethylene glycol; water-soluble acrylics; water-soluble polyesters; polyacryl amides; polyamines; polyquaternary amines; styrene maleic anhydride (SMA) resin; polyethylene amine; from Covestro, Baycusan® C 2000 (Polyurethane solution in ethanol (INCI-Polyurethane-64)), Baycusan® eco E 1000 (water-based polyurethane), Baycusan® C 1010 (water-based polyurethane), Baycusan® C 1008 (water-based polyurethane), Baycusan® C 1001 (water-based polyurethane); from Inolex, Inolex Lexfilm Sun (INCI-Polyester-7 (and) Neopentyl Glycol Diheptanoate), Inolex Lexfilm Sun Natural MB (INCI-Capryloyl Glycerin/Sebacic Acid Copolymer), Inolex WetFilm MB (INCI-Trimethylpentanediol/Adipic Acid/Glycerin Crosspolymer), Inolex Lexfilm Spray (INCI-Polyester-10 (and) Propylene Glycol Dibenzoate) and Inolex Lexorez 100 MB (INCI-Adipic Acid/Diglycol Crosspolymer); and other conventional polymers that are polar solvent soluble or that can be made soluble through neutralization with the appropriate base.

In one embodiment, the hair fixing formulation comprises at least one biodegradable starch ester-based polyglucose film-forming polymer in addition to the polyvinyl alcohol described herein.

In another embodiment, the hair fixing formulation comprises at least one biodegradable polyester film-forming polymer in addition to the polyvinyl alcohol described herein.

When the hair fixing formulation comprises another film-forming polymer in addition to the polyvinyl alcohol described herein, such other film-forming polymer may also be present in the hair fixing formulation in an amount of 0.1 to 10 wt % based on a total weight of the formulation.

In a preferred embodiment, such other film-forming polymer is incorporated into hair fixing formulations in an amount of 0.5 to 5 wt % based on a total weight of the formulation.

In another preferred embodiment, the combined amounts of all other such film-forming polymers and the polyvinyl alcohol described herein total 0.1 to 10 wt % based on a total weight of the formulation.

In an especially preferred embodiment, the combined amounts of all other such film-forming polymers and the polyvinyl alcohol described herein total 0.5 to 5 wt % based on a total weight of the formulation.

The disclosed hair fixing formulations may contain a wide range of additional, optional components which are referred to herein as “cosmetic components”, but which can also include components generally known as pharmaceutically active agents. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present disclosure. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), SPF boosters, waterproofing agents, and viscosity increasing agents (aqueous and nonaqueous).

It has been found that the polyvinyl alcohol polymers can provide hair fixative polymers that are not only biodegradable but that can also provide equal to or better hair styling performance for example with respect to spray rate, viscosity, and stiffness, especially as hair spray polymers, at comparable costs to their non-biodegradable alternatives. The inventive polymers are soluble in an alcohol based solvent system and, optionally, they are also compatible with hair styling propellants.

As is known in the art, solution viscosity is an important property of aerosol hair fixatives, where the balancing of aerosol spray characteristics (affected by solution viscosity) versus on-hair performance of the hair fixative (affected by the presence of water) presents a problem in low VOC hair fixatives. Having a low viscosity allows the polymer solution to be sprayed from solution and provides uniform coverage and small droplet size. The viscosity of a 5 percent polymer solids solution of the optionally partially-neutralized polymer in a solvent system including water and ethylene glycol dimethyl ether (EGDME) at relative weight percent of 67/33 and at 250° C. preferably will range from about 2 to about 10 cp, more preferably from about 2 to about 7 cp, even more preferably less than 5 cps.

In an embodiment, the polyvinyl alcohol polymers of the present disclosure may be present in the hair fixative composition in an amount from about 1 weight percent to about 10 weight percent, based on the weight of the hair fixative composition. In another embodiment, the polyvinyl alcohol polymers are present in an amount from about 2 weight percent to about 8 weight percent. In yet another embodiment, the polyvinyl alcohol polymers are present in an amount from about 2 weight percent to about 6 weight percent.

In an embodiment of the disclosure, the hair fixative composition will include no more than about 80% volatile organic compounds (VOC), such as alcohol and/or propellant with the remainder of the solvent being water. In another embodiment, the hair fixative compositions will comprise no more than about 55% volatile organic compounds.

In an embodiment of the disclosure, the hair fixative compositions optionally further include at least one neutralizing agent. Suitable basic neutralizing agents compatible with the composition can be employed, even inorganic materials such as sodium or potassium hydroxide. Generally organic amines or alkanolamines are readily used for neutralization. In an embodiment, the neutralizing agents include, but are not limited to ammonia; primary, secondary and tertiary amines; alkanolamines; and hydroxyamines such as 2-amino-2-methyl-propanol and 2-amino-2-methyl-1,3-propanediol, mono-, di-, and tri-long chain fatty amines containing a C₄ to C₂₄ hydrocarbon chain, ethoxylates and propoxylates long chain (C₄ to C₂₄) fatty amines and mixtures thereof. In another embodiment, the neutralizing agents include aminomethylpropanol, and di-methyl stearamine, inorganic materials, such as sodium hydroxide and potassium hydroxide, and triethanolamine. In an embodiment of the disclosure, the neutralizing agent is an organic amine or alkanolamine. In an embodiment, combinations of neutralizing agents may also be used.

In an embodiment, the hair fixative compositions have spray rate in a range from about 0.3 to about 1.5 grams/sec. in a >95% VOC system at 3% solids and 40% DME, in another embodiment, preferably from about 0.5 grams/sec. to about 1.2 grams/sec., and in another embodiment, more preferably from 0.7 about to 0.9 grams/sec.

Further optional cosmetically acceptable additives may also include one or more additives selected from the group consisting of hair fixative polymers, plasticizers, UV absorbers, dyes, perfumes, preservatives, viscosity modifiers, vitamins, moisturizers, anti-itch, and anti-dandruff ingredients. Mixtures of these optional additives may also be included. As previously noted, the polymeric binders of this disclosure show little or no tendency to interact with such additives adversely chemically.

The disclosure will now be described in greater detail with reference to the following non-limiting examples.

### Examples

### Example 1: Effect of Degree of Hydrolysis on Solubility

Attempts were made to solubilize various polymers of varying degrees of hydrolysis as indicated in Table 1 in a formulation comprising 63% anhydrous ethanol/37% organics. The desired result is a clear, one-phase, and complete solution, which is taken as an indication that the formulation is fully soluble. On the other hand, if the solution is hazy or contains precipitate, this is taken as an indication that the formulation is at least partly insoluble. Evaluations are made immediately after mixing (“Initial Solubility”) and 24 hours later (“24 Hour Solubility”).

TABLE 1

| | Film-Forming | Degree of | Initial | 24 Hour |
|---|---|---|---|---|
| # | Polymer | Hydrolysis (%) | Solubility | Solubility |
| 2 | Poval ™ LM-10 HD¹ | 38-42 | Clear | Clear |
| 3 | Poval ™ LM-20² | 38-42 | Clear | Clear |
| 4 | Active 35³ | 32.7 | Clear | Clear |
| 5 | Active 45⁴ | 44-46 | Clear | Clear |
| 6 | Active 55⁵ | 55.5-57.5 | Clear | Clear |
| 7 | Ethapol XS⁶ | 66.7 | Slight Haze | Precipitate |
| 8 | Other PVOH⁷ | 70-99.9 | Insoluble | Insoluble |

^{1,2}Polyvinyl alcohol available from Kuraray
^{3,4,5,6}Polyvinyl alcohol available from Nouryon
⁷We tested (1) Poval ™ 5-74 (DH = 72.5-74.5%); (2) Poval ™ 4-88 (DH = 86.7-88.7); and (3)
Poval ™ 4-98 (DH = 98.0-98.8%), and in each case the polymers were completely insoluble at 5 wt %, 2.5 wt %, and 1 wt % in anhydrous ethanol.

The results demonstrate a clear correlation between the degree of hydrolysis of the film-forming polymer and the solubility of the formulation in anhydrous ethanol.

Generally speaking, the solubility of any disclosed film-forming polymer in any particular solvent or solvent system can be determined analogously to this example in a simple and straightforward manner.

### Example 2

Derivatives were prepared as indicated in Table 2 and tested analogously to Example 1.

The succinates were prepared by reacting the base polyvinyl alcohol polymer with succinic anhydride.

The caprolactone condensation products were prepared by reacting the base polyvinyl alcohol polymer with caprolactone.

The vinyl pivalates were prepared in the manner described in Example 11 below.

The 2-ethylhexanoates were prepared in the manner described in Example 10 below.

The vinyl laurates were prepared in the manner described in Example 9 below.

TABLE 2

| | | Initial | 24 Hour |
|---|---|---|---|
| # | Film-Forming Polymer | Solubility | Solubility |
| 9 | PVA LM-20 Succinate (80:20) | Clear | Clear |
| 10 | PVA LM-20 Succinate (70:30) | Clear | Clear |
| 11 | PVA LM-10HD Succinate (80:20) | Clear | Clear |
| 12 | PVA LM-10HD Succinate (70:30) | Clear | Clear |
| 13 | PVA LM 10HD + Caprolactone (80:20) | Clear | Clear |
| 14 | PVA LM 10HD + Caprolactone (90:10) | Clear | Clear |
| 15 | PVA LM-20 + Caprolactone (90:10) | Clear | Clear |
| 16 | PVA LM-20 + Caprolactone (80:20) | Clear | Clear |
| 17 | PVOH Vinyl Pivalate (93:7; DH 32%) | Clear | Clear |
| 18 | PVOH Vinyl Pivalate (93:7; DH 55%) | Clear | Clear |
| 19 | PVOH Vinyl Pivalate (93:7; DH 73%) | Clear | Clear |
| 20 | PVOH Vinyl Pivalate (93:7; DH 83%) | Clear | Clear |
| 21 | PVOH Vinyl 2-Ethylhexanoate | Clear | Clear |
| | (95:5; DH 19%) | | |
| 22 | PVOH Vinyl 2-Ethylhexanoate | N.D. | N.D. |
| | (95:5; DH 36%) | | |
| 23 | PVOH Vinyl 2-Ethylhexanoate | N.D. | N.D. |
| | (95:5; DH 52%) | | |
| 24 | PVOH Vinyl 2-Ethylhexanoate | Clear | Clear |
| | (95:5; DH 73%) | | |
| 25 | PVOH Vinyl Laurate (96:4; DH 35%) | Clear | Clear |
| 26 | PVOH Vinyl Laurate (96:4; DH 61%) | Clear | Clear |
| 27 | PVOH Vinyl Laurate (96:4; DH 80%) | Clear | Clear |

N.D. = not determined

### Example 3

Selected polymer samples (using the numbering in the tables above) were prepared in ethanol solvent and blended with propellant according to the following procedure to understand compatibility with propellants.

### Formulation Preparation Procedure:

1. Charge all ethanol in the formulation to the main mixing vessel.
2. Begin mixing with propeller agitation (adjust the speed of the speed of the mixing until there is a vortex pulled ⅔ of the way down the mixing shaft).
3. Slowly add the polymer powder by sifting it into the side of the vortex. Allow the polymer to disperse completely.
4. Fill concentrate into aerosol containers and charge with propellant.
5. Charged aerosols are observed for clarity, phases, and any precipitation that may have formed initially and over time.

In the following examples the propellant is dimethyl ether (DME) at 30% or 40% of the total formulation. Where the propellant is 30 wt % DME, the test formulation comprised 3 wt % polymer, up to 1 wt % aminomethylpropanol, ethanol QS to 70 wt %, and 30 wt % dimethylether propellant. Where the propellant is 40 wt % DME, the test formulation comprised 3 wt % polymer, up to 1 wt % aminomethylpropanol, ethanol QS to 60 wt %, and 40 wt % dimethylether propellant.

The desired result is a clear, one-phase, and complete solution, which is taken as an indication that the formulation is compatible. On the other hand, if the solution is hazy or opaque or contains precipitate, this is taken as an indication that the formulation is at least partly incompatible.

TABLE 3

| # | Film-Forming Polymer | 30% DME¹ | 40% DME |
|---|---|---|---|
| 2 | Poval ™ LM-10 HD | Clear | Clear |
| 3 | Poval ™ LM-20 | Clear | Clear |
| 4 | Active 35 | Incompatible | Incompatible |
| 5 | Active 45 | Borderline | Borderline |
| 6 | Active 55 | Incompatible | Incompatible |
| 7 | Ethapol XS | Incompatible | Incompatible |
| 9 | PVA LM-20 Succinate (80:20) | Clear | Clear |
| 10 | PVA LM-20 Succinate (70:30) | Clear | Clear |
| 11 | PVA LM-10HD Succinate (80:20) | Clear | Clear |
| 12 | PVA LM-10HD Succinate (70:30) | N.D. | Incompatible |
| 13 | PVA LM 10HD + Caprolactone | Clear | Clear |
| | (80:20) | | |
| 14 | PVA LM 10HD + Caprolactone | Clear | Clear |
| | (90:10) | | |
| 15 | PVA LM-20 + Caprolactone (90:10) | Clear | Clear |
| 16 | PVA LM-20 + Caprolactone (80:20) | Clear | Clear |
| 17 | PVOH Vinyl Pivalate (93:7; DH | Clear | Clear |
| | 32%) | | |
| 18 | PVOH Vinyl Pivalate (93:7; DH | Clear | Clear |
| | 55%) | | |
| 19 | PVOH Vinyl Pivalate (93:7; DH | Clear | Clear |
| | 73%) | | |
| 20 | PVOH Vinyl Pivalate (93:7; DH | Clear | Clear |
| | 83%) | | |
| 21 | PVOH Vinyl 2-Ethylhexanoate | Clear | Clear |
| | (95:5; DH 19%) | | |
| 22 | PVOH Vinyl 2-Ethylhexanoate | Clear | Clear |
| | (95:5; DH 36%) | | |
| 23 | PVOH Vinyl 2-Ethylhexanoate | Clear | Clear |
| | (95:5; DH 52%) | | |
| 24 | PVOH Vinyl 2-Ethylhexanoate | Clear | Clear |
| | (95:5; DH 73%) | | |
| 25 | PVOH Vinyl Laurate (96:4; DH | Clear | Clear |
| | 35%) | | |
| 26 | PVOH Vinyl Laurate (96:4; DH | Clear | Clear |
| | 61%) | | |
| 27 | PVOH Vinyl Laurate (96:4; DH | Clear | Clear |
| | 80%) | | |

¹dimethyl ether (DME)
N.D. = not determined

The results show that solubility of the base polymer in anhydrous ethanol does not guarantee compatibility with propellants. However, a number of the disclosed film-forming polymers were compatible with 30% and 40% DME.

Generally speaking, the compatibility of any disclosed film-forming polymer and a particular propellant or solvent-propellant system can be determined analogously to this example in a simple and straightforward manner.

### Example 4

Several examples of this disclosure were tested by OECD biodegradability method 301D, details of which are readily available. Testing was performed in both river water and activated sludge.

TABLE 4

| | |
|---|---|
| | Biodegradability in River Water |
| Biodegradation | Film-Forming Polymer→ |

| percentage at | Poval ™ | |
|---|---|---|
| Day X↓ | LM-10 HD | Active 55 |
| 7 | 1 | 3 |
| 14 | 7 | 6 |
| 21 | 13 | 17 |
| 28 | 18 | 24 |
| 42 | 35 | 37 |
| 56 | 55 | 52 |
| 84 | 61 | 61 |

TABLE 5

| | |
|---|---|
| | Biodegradability in Activated Sludge |
| Biodegradation | Film-Forming Polymer→ |

| percentage | Poval ™ | |
|---|---|---|
| at Day X↓ | LM-10 HD | Active 55 |
| 7 | 1 | 5 |
| 14 | 1 | 10 |
| 21 | 12 | 15 |
| 28 | 21 | 24 |
| 42 | 38 | 42 |
| 56 | 43 | 57 |
| 84 | 44 | 62 |

The OECD 301D ratings include (1) “Ultimately Biodegradable,” meaning >60% biodegradable within a 60 day period; (2) “Readily Biodegradable,” meaning >60% biodegradation in 28 days or less; (3) “Inherently Biodegradable,” meaning 20% to 60% biodegradation in 28 days; and (4) “Non-Biodegradable,” meaning less than 20% biodegradation in 28 days.

### Example 5

Polymers according to the disclosure were tested in the Half-Head Wig Evaluation test for aerosol hair sprays.

### Equipment/Materials

Head, Sandy, Root turned manikin, #351, Bur Max Co.
Wig stand
Comb: Ace, black, hard rubber, 5″×1″ or equivalent
Test formula—hair spray to be evaluated
Control formula

### Testing Procedure

1) Place head on wig stand; comb hair to remove any tangles or snarls.
2) Using comb, divide wig into equal sections (left side and right side).
3) Place a divider in middle of head to protect each side from overspray.
4) Hold hair spray 6″ from hair and spray test formula for 10 seconds to one side of head, and 10 seconds of control formula to the other side of head. Let head dry 45 minutes at room temperature.
5) Spray two heads for each formulation to be tested (randomly alternate sides of test formula and control between the two heads).
6) Blind evaluations are then performed on each head by four panel members.
7) The panel member then evaluates a series of criteria and selects which side has more of each property (see below).
8) Differences in a performance property between the two samples are regarded as “no significant difference” when chosen two to six times out of eight; “significantly inferior” when chosen zero or one out of eight times, or “significantly superior” when chosen seven or eight out of eight times.

### Blind Panel Evaluations:

BEADING: Visually examine each side of the head for dried polymer beads. Choose the side which has more beading.
GLOSS: Visually inspect both sides of wig for degree of gloss. Determine which side has more shine/gloss.
STIFFNESS: Gently handle each side of head and “feel” for differences in stiffness. Choose which side is more rigid or stiff.
SPRING: While holding a hair bundle in one hand, gently pull on an edge with the other hand three times only. Look for spring back, and bounce. Select the side that has more elasticity and more Spring.
WEBBING: While holding a hair bundle in both hands, gently pull outward on each of the edges approx. 4″. (Do this three times only to avoid damage to the bonds). The more net-like the better the Webbing.
FEEL: Run fingers down each side of the head and determine preference. Choose the side that feels more silky/cleaner feel.
   Valve Specifications for Aerosol Hair Spray Testing (procured from Aptar)
PRODUCT: VX-81 229 mm
HOUSING: VX BARB ARIANE—0,33-0,00-PA
VALVE STEM: VX80-4,03-1-0,33-8,70-NATU-1
VALVE GASKET: BUT U133-2,54-8,00-1,14-BUT
SPRING: VX80—INOX—VX—OPEN COIL
VALVE FIXTURE: SUB-AL-CLCL-DR-32,64—S-GA
DIP TUBE: PE-NATU-4,07-3,10-RL—0.122 STD
   Actuator Specification (from Aptar)
PRODUCT: XL200 3302-05480-23
ACTUATOR: XL200-PP—3,90-WHIT-POLISH-SPRAY
INSERT: MS23—POM-BLAC RBK000568—MS.023 MISTY BLACK

### Testing and Results

Polymers according this disclosure were formulated at 3% polymer, 40% DME and remainder ethanol and subjected to testing and compared to Amphomer® as a control. The results are shown in Table 6 below.

### Analysis of Data/Assessments:

Sample exhibits less of the attribute: experimental selected 0 to 1 times out of 8 showing a statistically significant difference from the reference at a 95% confidence level
Sample shows no statistical difference to standard: experimental sample selected 2 to 6 times out of 8
Sample exhibits more of the property: experimental selected 7 to 8 times out of 8 showing a statistically significant difference from the reference at a 95% confidence level

With the exception of beading, it is desirable to have more of the property.

TABLE 6

| | Film-Forming Polymer | Beading | Gloss | Stiffness | Spring | Webbing | Feel |
|---|---|---|---|---|---|---|---|
| | Poval ™ LM-10 HD | 7 | 4 | 1 | 2 | 8 | 8 |
| | PVA LM-20 Succinate | 4 | 2 | 4 | 7 | 6 | 4 |
| | (80:20) | | | | | | |
| | PVA LM-20 Succinate | 3 | 2 | 0 | 1 | 3 | 7 |
| | (70:30) | | | | | | |
| | PVA LM-10HD Succinate | 4 | 1 | 4 | 6 | 6 | 1 |
| | (80:20) | | | | | | |
| 17 | PVOH Vinyl Pivalate (93:7; | 0 | 4 | 0 | 0 | 0 | 6 |
| | DH 32%) | | | | | | |
| 18 | PVOH Vinyl Pivalate (93:7; | 2 | 1 | 2 | 4 | 6 | 2 |
| | DH 55%) | | | | | | |
| 19 | PVOH Vinyl Pivalate (93:7; | 0 | 3 | 2 | 6 | 6 | 8 |
| | DH 73%) | | | | | | |
| 20 | PVOH Vinyl Pivalate (93:7; | 0 | 1 | 7 | 7 | 8 | 4 |
| | DH 83%) | | | | | | |
| 21 | PVOH Vinyl 2- | 0 | 0 | 1 | 2 | 3 | 6 |
| | Ethylhexanoate (95:5; DH | | | | | | |
| | 19%) | | | | | | |
| 22 | PVOH Vinyl 2- | 3 | 1 | 3 | 3 | 7 | 4 |
| | Ethylhexanoate (95:5; DH | | | | | | |
| | 36%) | | | | | | |
| 23 | PVOH Vinyl 2- | 1 | 3 | 3 | 3 | 7 | 2 |
| | Ethylhexanoate (95:5; DH | | | | | | |
| | 52%) | | | | | | |
| 24 | PVOH Vinyl 2- | 1 | 1 | 3 | 5 | 7 | 5 |
| | Ethylhexanoate (95:5; DH | | | | | | |
| | 73%) | | | | | | |
| 25 | PVOH Vinyl Laurate (96:4; | 3 | 2 | 8 | 8 | 8 | 3 |
| | DH 35%) | | | | | | |
| 26 | PVOH Vinyl Laurate (96:4; | 7 | 5 | 8 | 8 | 8 | 0 |
| | DH 61%) | | | | | | |
| 27 | PVOH Vinyl Laurate (96:4; | 5 | 1 | 5 | 8 | 7 | 3 |
| | DH 80%) | | | | | | |

It is clear from the foregoing that we are able surprisingly to achieve with the disclosed polymers performance on hair equal to and in many cases superior to the commercial standard.

### Example 6: Preparation of Intermediate Copolymer with Pendent Acetate Functionality; Precursor to PVOH Copolymers Esterified with Lauric Acid

A four-neck 1 L round bottom flask was equipped with a mechanical stirrer; a Claisen adapter fitted with a temperature probe and a reflux condenser; a 500 mL addition funnel (not pressure equalized); and a 125 mL addition funnel (not pressure equalized). To the flask was charged 7.50 g vinyl laurate, 67.5 g vinyl acetate, 51.15 g ethyl acetate, and 2.91 g 98% tert-butyl peroctoate. To the 500 mL addition funnel was charged a mixture of 22.50 g vinyl laurate and 202.50 g vinyl acetate (Monomer SA-1). To the 125 mL addition funnel was charged a mixture of 2.91 g 98% tert-butyl peroctoate and 67.95 g ethyl acetate (Initiator SA-2).

The reaction mixture was brought to reflux with stirring using a hot water bath. After the reaction mixture was at reflux for 5 minutes, the uniform addition of the contents of Monomer SA-1 to the reaction mixture over 4 h. was started. When Monomer SA-1 addition was complete, the addition funnel was flushed (into the reactor) with 4.61 g ethyl acetate. After the reaction mixture was at reflux for 1 h., the uniform addition of the contents of Initiator SA-2 to the reaction over 5 h. was started.

After the addition of Initiator SA-2 was complete, the reaction was held at reflux for an additional 2 h. (total reaction time to this point: 8 h. The reaction was then allowed to cool below reflux, and 70.0 g ethanol was added. After a uniform solution was obtained, the reaction was allowed to stand overnight.

After standing overnight, the reaction was brought back to reflux, and a solution of 0.90 g tert-butyl peroctoate in 7.50 g ethanol was added in a single shot. Heating at reflux was continued for 2 h. after the shot-wise addition of the initiator. The reaction was then allowed to cool below reflux, and 104.0 g ethanol was added. The reaction was stirred until a uniform polymer solution was obtained, and then it was allowed to cool. A clear, colorless viscous polymer solution was obtained. Solids (determined gravimetrically): 49.94%.

### Examples 7 and 8: Preparation of Intermediate Copolymers with Pendent Acetate Functionality; Precursors to PVOH Copolymers Esterified with 2-Ethylhexanoic Acid and Pivalic Acid, Respectively

Additional intermediate copolymers were prepared following the general procedure described in Example 6 using different hydrophobic esters of vinyl alcohol. The compositions of all intermediate copolymers, as estimate by the feed ratio of the two monomes, are summarized in Table 7 below.

TABLE 7

| | Monomer 1 | Monomer 2 |
|---|---|---|
| Intermediate | (wt. %) | (wt. %) |
| Example 6 | Vinyl acetate | Vinyl laurate |
| | (90) | (10) |
| Example 7 | Vinyl acetate | Vinyl 2- |
| | (90) | ethylhexanoate |
| | | (10) |
| Example 8 | Vinyl acetate | Vinyl pivalate |
| | (90) | (10) |

### Example 9: Partial Hydrolysis of Example 6 Intermediate Copolymer to Give PVOH Polymers Esterified with Lauric Acid

A four-neck 1 L round bottom flask was equipped with a mechanical stirrer; a Claisen adapter fitted with a temperature probe and a reflux condenser-topped Barrett trap; a septum; and a stopper. To the flask was charged 250.5 g of the polymer solution prepared in Example 6 (125.0 g, solids basis) and 146 g ethanol. The resulting mixture was stirred until a homogeneous solution was obtained and then 2.25 g of 98% sulfuric acid was added to the reaction mixture. The reaction was brought to a vigorous reflux using a hot water bath, and a total of 40 g of ethanol was distilled from the reaction via the Barrett trap. Heating was continued at mild reflux (no more distillation of ethanol) for 7 h. At 2 h. and 4.5 h. reaction time, about 65 g of the reaction mixture was withdrawn, stirred with about 4.5 g Amberlyst A21 weak basic ion exchange resin for about 1 h., filtered to remove the ion exchange resin, then pan-dried to a constant weight in a 60° C. forced air oven. A sample of the reaction mixture at the end of the reaction (at 7 h.) was similarly processed. In this manner, three partially hydrolyzed PVOH polymers esterified with lauric acid that differed in the degree of hydrolysis were obtained. The compositions are summarized in Table 8.

### Examples 10 and 11: Partial Hydrolysis of Example 7 and 8 Intermediate Copolymers to Give PVOH Polymers Esterified with 2-Ethylhexanoic Acid and Pivalic Acid, Respectively

The precursor polymers from Examples 7 and 8 were partially hydrolyzed following the same general procedure described in Example 9. The compositions as determined by ¹H NMR (400 MHz; DMSO-d₆ solvent) except where otherwise noted are summarized in Table 8 below.

TABLE 8
Hydrophobic acid used to esterify partially hydrolyzed PVOH: Lauric acid

| | Monomer → | | | | | | | | |
|---|---|---|---|---|---|---|---|---|---|
| | Hydrolysis | | Vinyl laurate | | Vinyl Acetate | | Vinyl Alcohol | | |
| Film-Forming | Reaction | | wt. | mole | wt. | mole | wt. | mole | DH |
| Polymer #↓ | Time | | % | % | % | % | % | % | (%) |
| 25 | 2 | h | 11 | 4 | 75 | 71 | 14 | 25 | 26 |
| 26 | 4.5 | h | 13 | 4 | 59 | 50 | 28 | 46 | 48 |
| 27* | 7 | h | 14 | 4 | 43 | 33 | 42 | 63 | 66 |

Hydrophobic acid used to esterify partially hydrolyzed PVOH: 2-Ethylhexanoic acid

| | Monomer → | | | | | | | | |
|---|---|---|---|---|---|---|---|---|---|
| | Hydrolysis | | Vinyl 2-ethylhexanoate | | Vinyl Acetate | | Vinyl Alcohol | | |
| Film-Forming | Reaction | | wt. | mole | wt. | mole | wt | mole | DH |
| Polymer # ↓ | Time | | % | % | % | % | % | % | (%) |
| 21 | 1.5 | h | 11 | 5 | 80 | 78 | 9 | 17 | 18 |
| 22 | 3 | h | 12 | 5 | 72 | 66 | 16 | 29 | 31 |
| 23 | 5 | h | 13 | 5 | 60 | 50 | 28 | 45 | 47 |
| 24 | 7 | h | 14 | 5 | 43 | 32 | 43 | 63 | 66 |

Hydrophobic acid used to esterify partially hydrolyzed PVOH: Pivalic acid

| | Monomer → | | | | | | | |
|---|---|---|---|---|---|---|---|---|
| | Hydrolysis | Vinyl Pivalate | | Vinyl Acetate | | Vinyl Alcohol | | |
| Film-Forming | Reaction | wt. | mole | wt. | mole | wt. | mole | DH |
| Polymer # ↓ | Time | % | % | % | % | % | % | (%) |
| 17 | 2 h | 11 | 7 | 76 | 70 | 13 | 23 | 25 |
| 18 | 4 h | 12 | 7 | 63 | 53 | 24 | 40 | 43 |
| 19 | 6 h | 14 | 7 | 47 | 35 | 40 | 58 | 62 |
| 20* | 8 h | 15 | 7 | 36 | 25 | 50 | 68 | 73 |

*Composition determined by Carbon-13 NMR (DMSO-d₆ solvent)

### Example 12: Influence of Polyvinyl Alcohol with Internal Gamma-Lactone (Cyclic Ester) Functionality on High Humidity Curl Retention

A series of intermediate copolymers with carboxylic acid pendent from the backbone—precursors to PVOH copolymers modified with an internal gamma-lactone (cyclic ester)—were prepared by copolymerizing vinyl acetate with crotonic acid in varying relative amounts following the general procedure described in Example 6. The compositions of these intermediate copolymers as determined by Carbon-13 NMR (DMSO-d₆ solvent) are summarized in Table 9 below.

TABLE 9

| | Monomer → | |
|---|---|---|
| Intermediate | Vinyl Acetate | Crotonic Acid/ |

| copolymer #↓ | wt. % | mole % | wt. % | mole % |
|---|---|---|---|---|
| 34 | 92 | 92 | 8 | 8 |
| 35 | 94 | 94 | 6 | 6 |
| 36 | 96 | 96 | 4 | 4 |
| 37 | 98 | 98 | 2 | 2 |

The intermediate (precursor) copolymers were partially hydrolyzed following the same general procedure described in Example 9. The compositions as determined by Carbon-13 NMR (DMSO-d₆ solvent) are summarized in Table 10 below. It should be noted that the majority of the carboxylic acid moieties pendent from the intermediate (precursor) copolymers spontaneously formed (cylic) esters (internal gamma-lactones) with alcohols pendent from the nascent PVOH copolymer under the conditions employed to partially hydrolyze the intermediate (precursor) copolymers; in some cases, a small fraction (<40%) of the carboxylic acid moieties pendent from the intermediate (precursor) copolymers did not form (cyclic) esters with alcohols pendent from the nascent PVOH copolymer.

TABLE 10

| | Monomer → | | | | | | | | | |
|---|---|---|---|---|---|---|---|---|---|---|
| | Vinyl Acetate | | Vinyl Alcohol | | Crotonic Acid/γ-Lactone | | Crotonic acid | | | |
| | wt. | mole | wt. | mole | wt. | mole | wt. | mole | DH | Tg |
| Copolymer #↓ | % | % | % | % | % | % | % | % | (%) | (° C.) |
| 28^{a} | 56 | 43 | 32 | 48 | 10 | 6 | 3 | 2 | 53 | 64.4 |
| 29^{a} | 59 | 48 | 27 | 43 | 11 | 7 | 2 | 2 | 47 | 62.8 |
| 30^{b} | 69 | 58 | 22 | 36 | 8 | 5 | 1 | 2 | 38 | 57.2 |
| 31^{c} | 67 | 54 | 27 | 42 | 5 | 3 | 1 | 1 | 44 | 53.2 |
| 32^{d} | 54 | 39 | 42 | 59 | 4 | 2 | 0 | 0 | 60 | 49.4 |
| 33^{d} | 61 | 46 | 35 | 52 | 4 | 2 | 0 | 0 | 53 | 50.8 |

^{a}Intermediate (precursor) polymer: 34.
^{b}Intermediate (precursor) polymer: 35.
^{c}Intermediate (precursor) polymer: 36
^{d}Intermediate (precursor) polymer: 37.

Hair spray formulations with polymers 29, 30, 31, and 32 were prepared as described in Example 3 with the exception that 95% ethanol (5% H₂O) was used as the solvent and then compared to similarly formulated Poval™ LM-20+5% H₂O (Kuraray) and Resyn™ 28-2930 anhydrous (Nouryon) in a high humidity curl retention test. Poval™ LM-20 has been discussed above. Resyn™ 28-2930 polymer is a film-forming hair fixative that offers cost-effective stiffness with flexibility in high alcohol hair spray formulas. It also provides strong holding power, manageability, gloss, and adhesion to the hair without flaking.

### Procedure for Determining High Humidity Curl Retention (HHCR)

The following procedures were used to conduct the evaluations of the objective performance of the polymers of the present disclosure as determined by high humidity curl retention. The high humidity curl retention properties of hair styling compositions including polymers of the present disclosure were measured. The tests were each conducted at 720° F. (22° C.) and 90% Relative Humidity over a period of 24 hours. The tests were performed on 10″ long×2-gram swatches of European virgin brown hair (9 replicate swatches per sample). Curl retention testing is run in a humidity chamber set at 70° F./90% Relative Humidity for a total of 24 hours. Readings for % Curl Retention are read and recorded at time intervals of 15, 30, 60, 90 min, 2, 3, 4, 5, and 24 hrs. The hair styling compositions were tested according to the following procedures:
1. Wet hair swatch, comb through to remove tangles and squeeze out excess water (run swatch between thumb and index finger).
2. Apply sample to swatch, gently “work into” swatch and comb through.
3. Roll swatch on ½″ diameter Teflon mandrel. Carefully remove rolled swatch from mandrel and secure with two hair clips.
4. Place curls on tray and dry in oven overnight.
5. Remove dried curls from oven and let cool to room temperature.
6. Suspend curls, from bound end of swatch, on graduated clear, transparent curl retention boards.
7. Remove clips from curls and gently unwind with glass rod making sure to “break” the curl.
8. Take initial curl length readings before placing boards and curls into environmental chamber (700° F., 90% relative humidity).
9. Record curl lengths at the 15, 30, 60, 90 min, 2, 3, 4, 5, and 24 hour time intervals.
10. At conclusion of test, remove boards and curls from chamber.
11. Clean used hair swatches.
12. Calculate % Curl Retention and comparison of samples.
   The Samples were Prepared as Follows:

HHCR was run in a constant temperature and humidity chamber. Curls were rolled on a mandrel and allowed to dry overnight. The curls were then sprayed with the polymer solutions (3 wt % polymer using 95% VOC ethanol-water and 40% DME) and allowed to dry. Then the curls were hung on a board placed in the oven and the percent of curl loss was tracked over 24 hrs.

The high humidity curl retention properties of hair styling compositions including polymers of the present disclosure according to the Examples as shown in Tables 11 and 12 that follow were measured and compared to the use of Poval™ LM-20 and Resyn™ 28-2930 polymers, respectively, in the same hair styling composition.

TABLE 11
High Humidity Curl Retention Compared to Poval ™ LM-20

| | Curl Retention Time in Humidity (hrs) | | | | | | | | |
|---|---|---|---|---|---|---|---|---|---|
| Polymer | 0.25 | 0.5 | 1 | 1.5 | 2 | 3 | 4 | 5 | 24 |
| 32 | = | = | + | + | + | + | + | + | + |
| 31 | = | = | + | + | + | + | + | + | + |
| 30 | + | + | + | + | + | + | + | + | + |
| 29 | = | + | + | + | + | + | + | + | + |

These data show the inventive polymers exhibited high humidity curl retention that was at least equal to that exhibited by Poval™ LM-20 at all times. Surprisingly and unexpectedly, all inventive samples provided statistically significant (at the 95% confidence level) superior high humidity curl retention from 1 hour onward. Surprisingly and unexpectedly, Polymers 29 and 30, which were exemplified by the highest tested levels of crotonic acid, achieved this statistically significant superiority beginning even earlier in time.

TABLE 12
High Humidity Curl Retention Compared to Resyn ™ 28-2930

| | Curl Retention Time in Humidity (hrs) | | | | | | | | |
|---|---|---|---|---|---|---|---|---|---|
| Polymer | 0.25 | 0.5 | 1 | 1.5 | 2 | 3 | 4 | 5 | 24 |
| Poval ™ | − | − | − | − | − | − | − | − | − |
| LM-20 | | | | | | | | | |
| 32 | = | = | = | = | = | = | = | − | − |
| 31 | = | = | = | = | = | = | = | − | − |
| 30 | = | = | = | = | = | = | = | = | − |
| 29 | = | = | = | = | = | = | = | = | − |

The foregoing data show that Poval™ LM-20 has statistically significantly inferior high humidity curl retention compared to Resyn™ 28-2930 as early as 15 mins. In contrast, surprisingly and unexpectedly, the inventive polymers are statistically equivalent to Resyn™ 28-2930 for the first 4 hours, with those having higher crotonic acid contents maintaining the equivalency for longer periods of time.

### Example 13

Polymers according this disclosure were formulated at 3% polymer, 40% DME and 5 parts water and remainder ethanol and subjected to testing and compared to Poval™ LM-20 as a control. The results are shown in Table 13 below.

Testing was conducted in the same manner as described in Example 5.

### Analysis of Data/Assessments:

Sample exhibits less of the attribute: experimental selected 0 to 1 times out of 8 showing a statistically significant difference from the reference at a 95% confidence level
Sample shows no statistical difference to standard: experimental sample selected 2 to 6 times out of 8
Sample exhibits more of the property: experimental selected 7 to 8 times out of 8 showing a statistically significant difference from the reference at a 95% confidence level

With the exception of beading, it is desirable to have more of the property.

TABLE 13
Half-Head Subjective Evaluations vs 3% POVAL ™
LM-20 + 5% water (All Samples Un-Neutralized)

| Polymer | Beading | Gloss | Stiffness | Spring | Webbing | Feel |
|---|---|---|---|---|---|---|
| 29 | 8/8 | 3/8 | 8/8 | 8/8 | 7/8 | 0/8 |
| 30 | 8/8 | 3/8 | 6/8 | 8/8 | 8/8 | 1/8 |
| 31 | 2/8 | 4/8 | 4/8 | 6/8 | 4/8 | 3/8 |
| 32 | 5/8 | 5/8 | 3/8 | 8/8 | 8/8 | 4/8 |

The value in each cell indicates the number of times the experimental sample was chosen over Poval™ LM-20 as having more of each attribute.

We observed the following trends in the data:
Higher beading as level of crotonic acid increased
Increasing stiffness as level of crotonic acid increased
Decreasing feel as level of crotonic acid increased
Gloss effectively independent of crotonic acid level
Spring and webbing—majority of inventive polymers tested were significantly better than Poval™ LM-20

### Example 14

Selected polymer samples having the composition in the table below were prepared in ethanol solvent and blended with propellant according to the procedure of Example 3 to understand compatibility with propellants.

In the following examples the propellant is HPC 152A at 40% of the total formulation. In each case, the test formulation comprised 3% polymer, 5% water, q.s. ethanol, and 40% hydrofluorocarbon 152A propellant.

The desired result is a clear, one-phase, and complete solution, which is taken as an indication that the formulation is compatible. On the other hand, if the solution is hazy or opaque or contains precipitate, this is taken as an indication that the formulation is at least partly incompatible.

TABLE 14

| | Monomer → | | | | | | | | | |
|---|---|---|---|---|---|---|---|---|---|---|
| | | | | | Crotonic Acid/ | | Crotonic | | Vinyl | |
| | Vinyl Acetate | | Vinyl Alcohol | | γ-Lactone | | Acid | | Pivalate | |
| | wt. | mole | wt. | mole | wt. | mole | wt | mole | wt. | mole |
| Copolymer #↓ | % | % | % | % | % | % | % | % | % | % |
| 38 | 59.18 | 48.00 | 27.11 | 43.00 | 11.24 | 7.00 | 2.47 | 2.00 | | |
| 39 | 44.89 | 38.00 | 26.58 | 44.00 | 13.85 | 9.00 | 2.36 | 2.00 | 12.31 | 7.00 |
| 40 | 39.16 | 32.00 | 31.92 | 51.00 | 14.35 | 9.00 | | | 14.58 | 8.00 |

In all cases, the copolymers proved to be compatible with the formulation, resulting in one-phase aerosols that were very slightly hazy.

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A hair fixing formulation comprising:
(a) a volatile solvent; and
(b) at least one film-forming polymer comprising at least one of: (i) polyvinyl alcohol or (ii) polyvinyl alcohol ester, said polyvinyl alcohol ester having ester functionality other than solely acetate, and (i) and (ii) being soluble in the volatile solvent, provided that if the at least one film-forming polymer comprises polyvinyl alcohol, then the polyvinyl alcohol has a degree of hydrolysis less than about 60%.

2. The hair fixing formulation according to claim 1, wherein the volatile solvent is an alcohol-based solvent system, wherein the alcohol-based solvent system comprises at least one C₁₋₆ straight or branched chain alcohol.

3. The hair fixing formulation according to claim 2, wherein the alcohol-based solvent system comprises ethanol.

4. The hair fixing formulation according to claim 1, which comprises 0-20% by weight of water.

5. The hair fixing formulation according to claim 1, which does not comprise water.

6. The hair fixing formulation according to claim 5, which comprises ethanol but not any other alcohol.

7. The hair fixing formulation according to claim 1, wherein the at least one film-forming polymer comprises polyvinyl alcohol having a degree of hydrolysis having a degree of hydrolysis of about 35% to about 58%.

8. The hair fixing formulation according to claim 1, wherein the at least one film-forming polymer comprises an ester of polyvinyl alcohol.

9. The hair fixing formulation according to claim 1, wherein the at least one film-forming polymer comprises partially hydrolyzed vinyl acetate-crotonic acid copolymer comprising pendant carboxylic acid groups and pendant hydroxy groups condensed together to form an internal gamma-lactone functionality.

10. The hair fixing formulation according to claim 9, wherein the at least one film-forming polymer comprises polyvinyl alcohol derivatized with 10-18 wt % crotonic acid based on 100 wt % of the at least one film-forming polymer, and the polyvinyl alcohol has a degree of hydrolysis of 35-50%.

11. The hair fixing formulation according to claim 1, which is a sprayable hair fixative formulation.

12. The hair fixing formulation according to claim 11, which further comprises one or more additives chosen from hair fixative polymers, plasticizers, UV absorbers, dyes, perfumes, preservatives, viscosity modifiers, vitamins, moisturizers, anti-itch, anti-dandruff ingredients, and combinations thereof.

13. The hair fixing formulation according to claim 12, which further comprises one or more additives chosen from film-forming polymers, plasticizers, UV absorbers, dyes, perfumes, preservatives, viscosity modifiers, vitamins, moisturizers, anti-itch ingredients, and combinations thereof.

14. The hair fixing formulation according to claim 1, which is an aerosol formulation, and further comprises a propellant.

15. The hair fixing formulation according to claim 14, wherein the propellant is dimethyl ether (DME).

16. The hair fixing formulation according to claim 14, which comprises a bag-on-valve device comprising an insert comprising the hair fixing formulation and propellant surrounding the insert.

17. A method of fixing hair comprising spraying hair with a hair fixing formulation according to claim 1 to hair.
